(19) United States
(12) Patent Application Publication
(10) Pub. No.: US 2004/0260387 A1
(43) Pub. Date: Dec. 23, 2004

(54) Drug-injecting stent for sustained and distributed drug delivery

(76) Inventors: Alan Chung Regala, Mountain View, CA (US); Kenneth Nicolas Horne, Palo Alto, CA (US); Larry Fan, San Francisco, CA (US)

Correspondence Address: Alan Regala, 65 Dalma Drive, Mountain View, CA, 94041, US

(21) Appl. No.: 10/854,083

(22) Filed: May 24, 2004

Related U.S. Application Data
(60) Provisional application No. 60/473,058, filed on May 23, 2003.

(51) Int. Cl. A61F 2/06

(52) U.S. Cl. 623/1.15

(57) ABSTRACT

A stent includes an expandable, generally tubular wall structure that can be radially expanded between a collapsed tubular configuration and an expanded and supportive tubular configuration. The stent defines a longitudinal axis. At least one penetrating projection (defining a projection axis) is attached to the wall of the stent. The penetrating projection supports a treatment drug and is pivotal between a stowed position, wherein the projection axis lies generally parallel to the longitudinal axis of the stent, and a penetrating position, wherein the projection axis lies generally perpendicular to the longitudinal axis of the stent and directed radially outward. The penetrating projection is in it's stowed position when the stent is in it's collapsed tubular configuration and moves to it's penetrating position when the stent expands to it's expanded and supportive tubular configuration. The length of the at least one penetrating projection is predetermined according to the particular lesion and is designed to deliver the treatment drug to a particular section of the artery wall to treat vulnerable plaque.

### CROSS-REFERENCE TO RELATED APPLICATIONS

This application claims priority from U.S. provisional application, Ser. no.: 60/473,058, filed May 23, 2003 and entitled: “Drug-Injecting Stent for Sustained and Distributed Drug Delivery”, the entire content of this provisional patent application being incorporated herein by reference.

### FIELD OF THE INVENTION

This invention generally relates to expandable intraluminal vascular grafts, commonly referred to as stents, and more particularly to such stents that include means to deliver and release a drug at the site of a lesion.

### BACKGROUND OF THE INVENTION

For many years, it was thought that the main cause of a heart attack or stroke was the buildup of fatty plaque within an artery leading to the heart or brain. With time, the plaque buildup would narrow the artery so much that the artery would either close off or become clogged by a blood clot (much like a clogged drain). The lack of oxygen-rich blood to the heart would then lead to a heart attack. However, these types of blockages cause only about 3 out of 10 heart attacks.

Researchers have learned that many people who have heart attacks do not have arteries severely narrowed by plaque. It has been found that inflammation within the artery walls can lead to the development of “soft” or vulnerable plaque. Although such vulnerable plaque typically lies within an artery wall and may not always bulge out and block the blood flow through the artery, this type of plaque is particularly dangerous because certain conditions within the body can cause it to encourage local blood clotting, which can thereafter disrupt blood flow.

Vulnerable plaque is first formed by fat droplets that are absorbed by the artery. This causes the release of proteins (called cytokines) that lead to inflammation. The cytokines make the artery wall sticky, which attracts immune-system cells (called monocytes). The monocytes squeeze into the artery wall. Once inside, they turn into cells called macrophages and begin to soak up the fat droplets. The fat-filled cells form a plaque with a thin covering. When this inflammation is combined with other stresses, like high blood pressure, it can cause the thin covering over the plaque to crack and bleed, spilling the contents of the vulnerable plaque into the bloodstream. The sticky cytokines on the artery wall capture blood cells (mainly platelets) that rush to the site of injury. When these cells clump together, they can form a clot large enough to block the artery.

Stents are expandable tubular devices that are implanted within a body's vessels in an effort to prevent collapse of the vessel and/or impede restenosis. Implantation of a stent is typically accomplished by first mounting the stent onto an expandable portion of a balloon-tipped catheter. The catheter is then maneuvered through the vasculature of a patient to position the stent at a desired location within the body lumen. Once in position the balloon portion of the catheter is inflated, which directly causes the stent to expand into engagement with the lumen wall. Typically, such stents automatically lock the expanded configuration which allows the balloon of the catheter to be deflated and the catheter removed from the body to complete the implantation procedure.

It is often desirable to provide localized pharmacological treatment of a vessel at the site being supported by the stent and it has been found convenient to utilize the stent as a delivery vehicle for such purpose. To this end, various stents have been provided with drug-coatings that are designed to slowly release at the site once the stent is expended in position.

One problem with such drug-coated stents stems from the tremendous mechanical strength and flexibility stents need to provide in order to pushback and support a vessel wall. Drug coatings must accommodate for this mechanical stress and movement of their supporting substrate (i.e., the stent structure) without breaking apart, but, at the same time, must also be sufficient in dose (e.g., thickness of the coating) to be effective in treating the lesion over a period of time. It is desirable for the drug to be released slowly and consistently over a period of time, in some cases, as long as six months or longer.

Studies have shown that injecting drugs using a single micro-syringe into the coronary adventitia, a section of the vessel wall, results in proximal and distal distribution throughout the entire circumference of the vessel. The distribution is enough that a single injection could span the greater coronary vasculature if properly placed. The downside of implementing this method during a procedure is that significant drug concentrations are gone four days after the injection and therefore have only limited initial affect.

The prior art has been unable to overcome such shortcomings and a new approach is needed for effectively delivering sufficient drug to a lesion site, in particular, a site of vulnerable plaque and thereafter applying the drug to the site over a prolonged period of time in an effort to treat vulnerable plaque and prevent secondary effects, such as heart attack and stroke.

### SUMMARY OF THE INVENTION

A stent includes an expandable, generally tubular wall structure that can be radially expanded between a collapsed tubular configuration and an expanded and supportive tubular configuration. The stent defines a longitudinal axis. At least one penetrating projection (defining a projection axis) is attached to the wall of the stent. The penetrating projection supports a treatment drug and is pivotal between a stowed position, wherein the projection axis lies generally parallel to the longitudinal axis of the stent, and a penetrating position, wherein the projection axis lies generally perpendicular to the longitudinal axis of the stent and directed radially outward. The penetrating projection is in it's stowed position when the stent is in it's collapsed tubular configuration and moves to it's penetrating position when the stent expands to it's expanded and supportive tubular configuration. The length of the at least one penetrating projection is predetermined according to the particular lesion and is designed to deliver the treatment drug to a particular section of the artery wall to treat vulnerable plaque. These and other features and advantages of the present invention will become apparent from the following detailed description of a preferred embodiment which, taken in conjunction with the accompanying drawings, illustrates by way of example the principles of the invention.

Other features and advantages of the present invention will become more apparent from the following detailed description of the invention. When taken in conjunction with the accompanying exemplary drawings.

### BRIEF DESCRIPTION OF THE DRAWINGS

FIG. 1 is a side view of a stent, according to the present invention, showing penetrating needles located in a collapsed orientation;

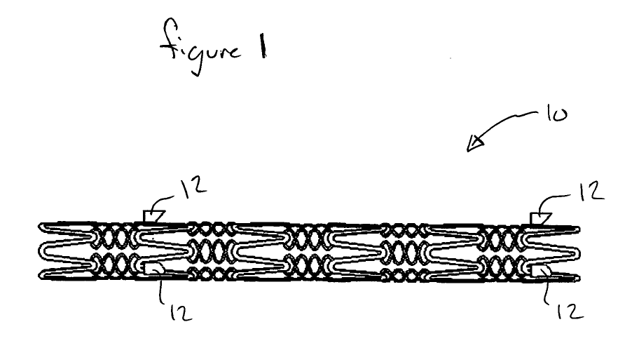

FIG. 2 is an enlarged view of a section of the stent of FIG. 1, showing details of two penetrating needles, according to the invention;

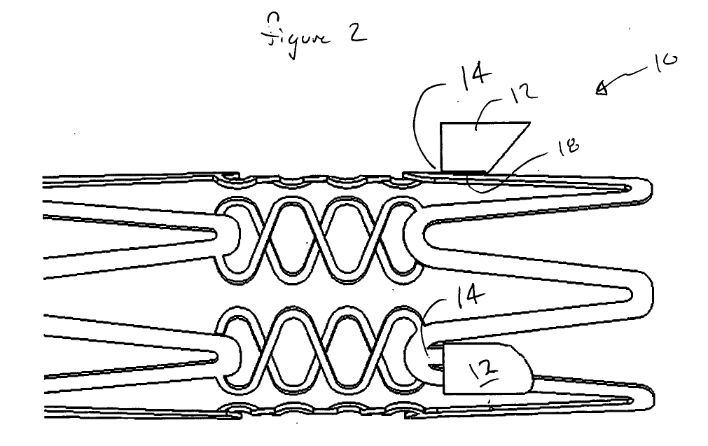

FIG. 3 is a side view of the stent of FIG. 1 showing the penetrating needles located in an upright and piercing orientation, according to the invention; and

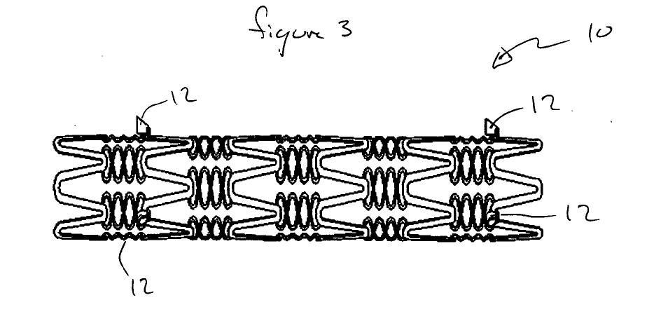

FIG. 4 is an enlarged view of a section of the stent of FIG. 3, showing details of two of the penetrating needles, according to the invention.

### DETAILED DESCRIPTION OF THE PREFERRED EMBODIMENTS

Adventitial drug delivery is an attractive method to treat vulnerable plaques because of the diffusive nature of the disease. Targeted drug delivery that spanned the coronary vasculature would allow administration of powerful drugs that are not target-specific and have them reach areas affected by vulnerable plaque. Further, these affected areas need not be located.

Referring to the figures, a drug-injecting stent 10 is shown, according to the invention. Regarding the radially-supportive function of the stent, stent 10 may be conventional and may follow the general stent structure shown in U.S. Pat. Nos. 6,733,524, 6,730,117, and 6,723,119, for example. The content of these prior art patents are incorporated by reference herein in their entirety as if they were reprinted within this specification. Regardless of the specific supportive structure of stent 10, stent 10 is generally tubular in shape, defining a longitudinal axis, and deformable from a collapsed (small-diameter tube) configuration to a deployed and expanded (larger-diameter tube) configuration. Such deployment from its collapsed to its expanded configurations may be in response to either a radially expanding force, such as that created by an inflating balloon (a balloon-tipped catheter), or by other means, such as through the use of well-known Nitinol alloy materials. Nitinol is a superelastic shape memory alloy made from nickel and titanium. When heat (such as body heat) is applied to Nitinol, the material automatically deforms to a predetermined expanded memory shape, as understood by those skilled in the art.

The basic concept behind the present invention resides in providing at least one penetrating needle 12 to the stent structure so that when the stent is located at the lesion site and in response to an applied radial force, the penetrating needle 12 will penetrate into the tissue of the arterial walls to a specific predetermined depth (or wall layer). The penetrating needle 12 includes a specific drug or drugs in either liquid, gel, solid or other form. The purpose of penetrating needle 12 is to deliver a therapeutic drug to a desired depth at the lesion site.

According to the invention, stent 10 includes at least one penetrating needle 12 which may be either open, like a trough, hollow, like a tube (such as a short hyperdermic needle with a sharpened beveled rim), or solid like a micro-spike. The size and shape of the penetrating member 12 is preferably sufficient and appropriate to carry a meaningful supply of treatment drug, yet not so large so as to cause irreversible trauma or tissue damage to the arterial wall. It is desirable to deliver a consistent and sustained dose of drug to a specific layer of the artery wall over a long period of time—a time-controlled release of drug to the local site.

The length of each penetrating needle 12 may be specific to the lesion being treated and the particulars of the patient. In other words, the penetrating needle may be offered in a variety of sizes (already attached to a stent 10) and the surgeon will select an appropriate size depending on what part of the arterial wall s/he wishes to treat or which layer of the arterial wall is best suited to receive the drug dose. One stent 10 may also have several penetrating needles 12, each of a different length.

Once at the desired layer within the arterial wall, the drug will spread within the tissue located around the artery and will further advance within the tissue around the artery along a length of the artery at the lesion site and for a distance on either side.

Regardless of the particular characteristics of penetrating needle 12, in each case the member is mounted to a portion of stent 10 is adapted to move in response to movement of stent 10 or in response to a remote activator, described below. According to a first embodiment of the invention, each penetrating needle 12 is mounted to an otherwise conventional stent structure in such a manner that the projection 12 lies generally parallel to the longitudinal axis of the tubular stent 10 when stent 10 is in it's collapsed configuration.

Penetrating needle 12 may be secured to the stent 10 anywhere along the length of the stent. The exact location will help control the point of entry into artery wall with respect to the lesion site. It may be desirable to administer drugs at the lesion site, ahead of the lesion site or just beyond the lesion site. Also, in addition to the supply of drug the actually penetrating needle 12 can carry within it's own structure, a reservoir may be provided in fluid communication with the penetrating needle 12 for allowing a greater supply of drug to be dispensed.

Each penetrating needle 12 is attached to the stent at a mounting point 14. Mounting point 14 can be made part of the stent itself, or an additional part made from Nitinol or stainless steel, for example. As described above, penetrating member 12 is preferably mounted to stent 10 in such a manner that keeps penetrating member generally parallel to the longitudinal axis of the stent until deployment. This would allow the stent structure to remain somewhat small in sectional area and therefore allow easy passage of the stent throughout the vasculature of the patient to the lesion site. The penetrating needles 12 would deploy only when stent 10 is at the lesion site.

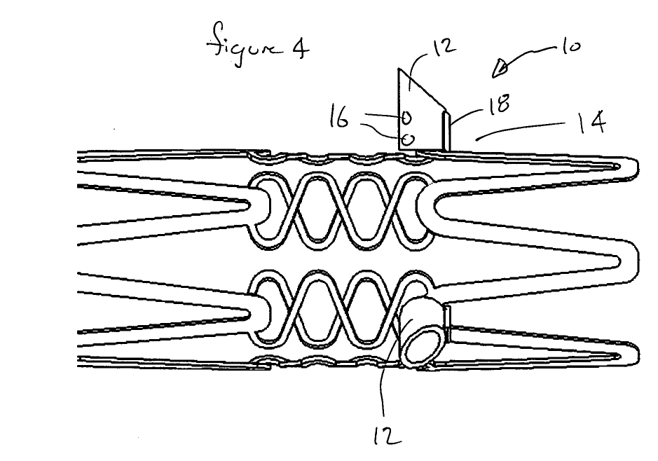

According to a first embodiment of the invention, referring to FIG. 4, the penetrating needle 12 could be a hollow needle and could include a variety of openings 16 along the sides of the needle so that drug could be delivered at different depths of the arterial wall once the penetrating needle 12 has advanced into the arterial wall. In this arrangement, as the stent 10 is expanded at the lesion site and each penetrating needle 12 is forced into the artery walls. Drug located within each penetrating needle 12 is release through openings 16, and also the opening at the end of the needle. As discussed above, a reservoir of additional drug may be provided on or within the stent 10 to supply additional drug to the penetrating needle 12 as needed.

According to another embodiment of the invention, stent 10 includes guide openings (not shown). Penetrating needles 12 are positioned within the body of the stent 10 prior to stent deployment and are aligned with the guide openings. As penetrating needles 12 are forced outward to their penetrating and extended position (by the inflating balloon, for example), they pass through the guide openings which help support and guide the needles 12 to ensure that they extend in the expected and desired orientation with respect to the stent body 10.

According to yet another embodiment of the invention, penetrating needles 12 are each secured to the stent 10 by a mounting tab 18 (shown in FIG. 4). Tabs 18 are initially flush with the surface of stent 10 and when deployed, tabs 18 bend or deform perpendicularly with respect to the surface of the stent 10 (i.e., directed radially outward). The tabs 18 can be spring biased to a perpendicular orientation and held flat against the stent 10 by a sheath or cover (not shown) so that during placement of the stent 10, the sheath or cover prevents the penetrating needles 12 or the mounting tabs 18 from engaging with the arterial wall tissue, but upon positioning the stent 10 at the lesion site, the cover or sheath is removed which allows the mounting tabs 18 to spring to their desired perpendicular orientation forcing the penetrating members to engage with the arterial wall tissue as desired. Alternatively, the tabs could be made of Nitinol and would expand when heated, either using a heating element in the catheter or by natural body heat.

The injection apparatus serves the important roll of delivering drug to the correct location, after a calculated delay and rate. Previous studies indicate success using micro-syringes that prevent vessel damage due to perforation. The injection apparatus could take two forms: a syringe, or an eluting needle.

Using a syringe makes it easy to control where the drug is released. The length of the syringe and the placement of the tip can be adjusted to insure drug delivery within the adventitia, desirable to spread therapeutic agents. Syringes of shorter lengths could also be employed to release anti-restenosis drugs into the two inner vessel layers. Delayed activation of the syringes could be achieved by coating the exit orifice with a biodegradable buffer, which would have dissolve before the drugs were released. By staggering release periods of the drugs, active therapy can be sustained.

An eluting needle eliminates the necessity of a reservoir. Although volumetrically less drugs could be utilized, the design is much simpler. In essence, the needle is an extension of the drug-coated stent. The needles could be coated with a different drug/polymer combination. As the polymer is degraded by the body, the drugs will be released locally. Adventitial delivery can be achieved by simply extending the needle into the adventitia. Portions of the needle intended to reach different layers of the vessel could be coated with different drug/polymer combinations to elute anti-restenosis drugs to the inner layers, and vulnerable plaque therapeutic agents into the adventitial layers.

A reservoir for the drugs could be necessary if the volume of drug desired is larger than that containable within the syringe, or if a way to facilitate drug expulsion is necessary. The reservoir could either be independent to each syringe, or coupled such that multiple needles share a single reservoir. If the reservoir were dynamic, it could react to the blood pressure fluctuations per heart beat and utilize the pressure wave to help expel drugs out the syringe.

The shelf life of the desired drug may be too short to attain effective drug delivery periods. Further, the desired concentration and volume of the drug may be infeasible to integrate into a single stent. This might entail crystallizing the drug, and storing that solid form within the reservoir, and having a mixing chamber before the syringe to allow the drug to return to liquid form.

Rather than use a passive method to sustain delivery, as described above, the reservoir could be externally activated to allow active drug delivery. Various activation methods such as high-frequency RF, high-intensity ultrasound, and even electric current activated silicon cells could be utilized. An activated drug delivery would allow a clinician to control drug release post-implantation, and base the injection cycles on patient condition.

While the invention has been illustrated and described herein in terms of its use as an intravascular stent, it will be apparent to those skilled in the art that the stent can be used in other instances such as to expand prostatic urethras in cases of prostate hyperplasia. Other modifications and improvements may be made without departing from the scope of the invention.

Other modifications and improvements can be made to the invention without departing from the scope thereof.

What is claimed is:

1. A longitudinally flexible stent for implanting in a body lumen, comprising:
a generally tubular body made up of a plurality of expansion arms and interposed openings and being expandable from a collapsed orientation to a radially expanded orientation;
at least one penetrating needle pivotally attached to one of said plurality of expansion arms of said tubular structure, said penetrating needle being pivotal between a collapsed position and an erect position, said penetrating needle being sized and shaped to penetrate the tissue walls of said body lumen a predetermined distance when in said erect position; and
wherein said at least one penetrating needle is in said collapsed position when said tubular body is in said collapsed orientation and moves to said erect position in response to said tubular body moving to said radially expanded orientation.
a plrality of cylindrical elements each having undulations, the cylindrical elements being expandable in the radial direction and interconnected to be generally aligned on a common longitudinal axis;
